(19) United States
(12) Patent Application Publication
(10) Pub. No.: US 2025/0268484 A1
(43) Pub. Date: Aug. 28, 2025

(54) PORTABLE NEONATAL MRI SYSTEM AND METHOD

(71) Applicant: The General Hospital Corporation, Boston, MA (US)

(72) Inventors: Clarissa Z. Cooley, Charlestown, MA (US); Lawrence L. Wald, Charlestown, MA (US); Jason Stockmann, Charlestown, MA (US); Sara Bates, Charlestown, MA (US)

(21) Appl. No.: 18/858,273

(22) PCT Filed: Apr. 20, 2023
(86) PCT No.: PCT/US2023/066026

§ 371 Date: Oct. 18, 2024

Related U.S. Application Data
(60) Provisional application No. 63/333,046, filed on Apr. 20, 2022.

(51) Int. Cl. A61B 5/055; A61B 5/00; G01R 33/383; G01R 33/563

(52) U.S. Cl. CPC A61B 5/055; A61B 5/704; G01R 33/383; G01R 33/56341; A61B 2503/045; A61B 2560/0431

(57) ABSTRACT

A system and method are provided that include a portable neonatal magnetic resonance imaging (MRI) system. The system includes a static (B₀) magnet comprising a plurality of magnetic elements arranged in a bulb array, a gradient coil, a patient bed movable relative to the B₀ magnet and one or more gradient coils, and an imaging radiofrequency coil. A method for manufacturing is provided in which the arrangement of magnetic elements is optimized to produce a target magnetic field within a target scanning volume.

### CROSS-REFERENCES TO RELATED APPLICATIONS

This application is based on, claims priority to, and incorporates herein by reference for all purposes, U.S. Provisional Patent Application No. 63/333,046 filed on Apr. 20, 2022.

### STATEMENT REGARDING FEDERALLY SPONSORED RESEARCH

This invention was made with government support under 5R01EB018976-04 and 1R01HD104649-01 awarded by the National Institutes of Health. The government has certain rights in the invention.

### BACKGROUND

The neonatal brain is particularly vulnerable to a wide variety of brain insults and injuries, such as birth asphyxia or Hypoxic-Ischemic Encephalopathy (HIE), meningitis, and stroke that can lead to lifelong neurological deficits and even death. For example, HIE affects 1-8 per 1000 live births in developed countries and up to 2.6% in resource-limited settings. The frequency and severity of HIE is higher in the preterm population (<37 weeks of gestation).

HIE is caused by asphyxia and oxygen deprivation to the brain during the perinatal period just before delivery. Neonates may present with poor muscle tone, low heart rate, slow or irregular respiration, bluish skin color, and low PH levels in the cord blood consistent with metabolic acidosis. HIE is a leading cause of mortality, accounting for 23% of neonatal deaths, and neurological morbidity in children. Surviving neonates with moderate to severe HIE may have shortened lifespans with decreased cognitive function, epilepsy, cerebral palsy, and other neurological deficits. Any success in treating these injuries results in a lifetime reduction in burden for the patients and costs to our healthcare and educational systems.

The pattern of HIE and resulting Hypoxic-Ischemic injury (HII) depends on the extent of oxygen deprivation and brain maturation. Thus, neuroimaging is essential to identify and characterize HII and to rule out hemorrhage. Sensitive methods to diagnose acute brain injury could enable early diagnosis, timely intervention, and critical guidance to treatment decisions, which can have profound effects on the long-term sequelae of brain injury in neonates, preventing further injury and potentially enhancing repair. The significance of successful clinical management of these young patients is amplified by the life-time integrated benefits of reducing disability.

These patients benefit from diffusion magnetic resonance imaging (MRI), but imaging must often be delayed until the patient is stable enough for transport to the MRI scanner. The time-sensitive need for imaging and the difficulty of transporting these sensitive patients to an MRI suite, or even across the neonatal intensive care unit (NICU), motivates the bedside point-of-care (POC) approach described herein. The availability of a POC MRI scanner in the NICU would have tremendous benefits for diagnostic and monitoring of neonatal brain injury.

Currently, ultrasound (US) is the primary modality for early neuroimaging and HIE assessment, followed by magnetic resonance imaging (MRI) when the patient is stable enough for the trip to the MRI suite. In neonates, the large space between the cranial sutures enables most of the brain to be imaged with US. US has the advantage of being inexpensive, portable, and non-radiation exposing, allowing routine and repeat screening.

US is able to detect large hemorrhages, hydrocephalus, and PVL cysts, but lacks the sensitivity and specificity of MRI for a number of reasons. The restriction of wave propagation through the cranial sutures and obstructions from scalp contact (e.g. IV leads, electroencephalogram (EEG) monitors, etc.) can lead to imaging “blind spots” and missed lesion detection. Furthermore, the lack of contrast mechanisms in US leads to non-specific lesion detection (e.g. hemorrhage vs. ischemia vs. edema). US is also insensitive to abnormalities in the cerebral convexity and brainstem. Finally, the hand-held nature of US makes it vulnerable to operator variability and is reliant on the availability of trained personnel. The latter makes off-hour imaging challenging.

CT can be useful for expedient screening for hemorrhaging without the need for sedation, but it has the major disadvantage of radiation exposure, which is particularly concerning in neonates. It is also less sensitive than MRI and US in HIE assessment. The poor soft-tissue contrast of CT is exacerbated because the high water content in the neonatal brain reduces contrast between normal and injured tissue.

MRI, particularly diffusion-weighted MRI (DWI), is unequivocally the most sensitive and specific imaging technique for HII evaluation. Together with conventional T₁ and T₂ weighted MRI, DWI can exclude other non-HIE causes of encephalopathy (hemorrhage, neoplasms, congenital malformations), and identify HIE markers earlier and more sensitively than US. DWI and apparent diffusion coefficient (ADC) maps show HIE markers of cytotoxic edema as restricted diffusion with very high sensitivity, making DWI the most sensitive HII evaluation tool. However, current MRI technologies face many barriers to use in the neonate population.

Aside from a few investigational scanners, MRI scanners are large, heavy, and expensive devices that are sited in dedicated shielded suites in a central radiology facility, usually remote from the NICU. The most significant challenge for neonatal MRI is the removal of the baby from the NICU and transport to the scanner, which raises safety concerns, including a dramatic disruption to care, remoteness from specialized emergency equipment, and environmental exposure. While safety is the number one concern, the burden on the hospital workflow is also challenging. It requires tremendous teamwork and coordination from multiple sources to transfer a critically ill neonate. For example, a neonatologist, nurse, and respiratory therapist must accompany the infant to the scanner and remain with the patient for the duration of the exam. It typically takes about three hours for the transfer to and from the NICU and the MRI scan, during which time replacement staff are needed in the NICU.

Acoustic noise is another problem with conventional neonatal MRI. For example, 1.5 T MRI echo planar imaging scans can produce sound pressure levels up to 131 dBA, but NICUs often restrict acoustic noise levels to about 45 dBA due to the stress and damaging effects on neurological development that can be caused by excessive noise. Therefore, hearing protection, such as ear plugs and earmuffs, is critical but is still unable to reduce the noise to these optimum limits. Not only does the noise cause stress to the patient, but it also reduces the potential to scan without sedation, which has well-known associated risks.

Critically ill neonates must move from their incubator/isolette in the NICU to a transport isolette that may or may not be MR compatible. The patient's care is significantly disrupted due to the removal/replacement of leads, tubes, ventilation equipment, temperature maintenance equipment, etc. Furthermore, intravenous pumps are not MR compatible, so extension tubing must be placed, which can interrupt the administration of critical medications. Even if “MR-safe”, non-compatible or metallic equipment can cause susceptibility artifacts, which are more severe at higher field strengths. Standard MRI hardware, such as coils, and sequences are not designed specifically for neonatal imaging and therefore yield sub-optimal results.

Thus, there remains a need for improved systems and methods for diagnosing and monitoring neonatal patient's brains to achieve high quality diagnostic information without exposing the vulnerable patients to extraneous risks.

### SUMMARY

The present disclosure overcome the aforementioned drawbacks by providing systems and methods for reduced-size movable or portable magnetic resonance imaging (MRI) systems that can be brought to neonatal patients, including in sensitive environments, such as the NICU. In accordance with one aspect of the present disclosure, a system for portable neonatal MRI is described in which the static main magnetic field (B₀) is generated by a static magnet of several magnetic elements arranged in an array. The system may further include a gradient coil and an imaging radiofrequency (RF) coil. The system may also include a patient bed that is movable relative to the B₀ magnet and the gradient coil to facilitate positioning of the neonate.

In accordance with another aspect of the present disclosure, a portable neonatal MRI system is provided that includes a static magnet and a gradient coil. The gradient coil may be positioned outside the static magnet or inside. The static magnet may be configured from several permanent magnetic elements arranged in an array. The system further includes a patient bed, which is movable relative to the B₀ magnet and gradient coil, and an imaging RF coil.

In accordance with yet another aspect of the present disclosure, a method for neonatal MRI is described in which a portable neonatal MRI system is transported to a bedside of a neonate. The portable neonatal MRI system may include a controller, a permanent B₀ magnet that includes several permanent magnetic elements arranged in a Halbach array, a gradient coil, a patient bed, and an imaging RF coil. The gradient coil may be arranged outside or inside the Halbach array. After transporting the system, the subject may be placed onto the patient bed and positioned such that the Halbach array partially surrounds the head of the subject. The controller can be used to control the gradient coil and imaging RF coil in order to acquire MRI data of the subject, and the data can be reconstructed to provide images of the subject.

In another aspect of the present disclosure, a method for manufacturing a neonatal MRI system is provided. To manufacture the system, a desired magnetic field is defined within a target scanning volume. An optimization can be performed over the target scanning volume in order to determine an arrangement of magnetic elements to optimally achieve the desired magnetic field based on a simulated magnetic field. The optimization may vary the positions, orientations, or sizes of each of the plurality of magnetic elements or may vary the total number of magnetic elements. Based on the optimization results, the magnetic elements may be optimally arranged within the array to produce a magnetic field. A gradient coil can further be included outside the Halbach array to provide a switchable gradient for imaging or image contrast manipulation. The desired magnetic field may be defined as a homogeneous B₀ field or a functionally linear magnetic field gradient over the target scanning volume. Additionally, the optimization may be constrained by a size or weight of the Halbach array to maintain portability and ease of use.

### DESCRIPTION OF THE DRAWINGS

FIG. 1A is an illustration of a portable MRI system according to one aspect of the present disclosure.

FIG. 1B is another illustration of the portable MRI system of FIG. 1A according to one aspect of the present disclosure.

FIG. 2A is an example arrangement of a Halbach array that produces a static magnetic field in accordance with one aspect of the present disclosure.

FIG. 2B is an example arrangement of a Halbach array that produces a static magnetic field and a permanent gradient in accordance with one aspect of the disclosure.

FIG. 3A is a block diagram outlining a manufacturing process for a portable neonatal MRI system in accordance with one aspect of the disclosure.

FIG. 3B is a graph showing part of an optimization of the Halbach array arrangement, in which a target field is defined in a target scanning volume and each magnetic element is modeled as a magnetic dipole to produce the desired field.

FIG. 4 illustrates example phase encoding gradient coils for use with the portable neonatal MRI system according to some aspects of the present disclosure.

FIG. 5A shows one configuration of a Transmit/Receive radiofrequency coil for use with the portable MRI system in accordance with the present disclosure.

FIG. 5B shows a further configuration of a Transmit/Receive radiofrequency coil for use with the portable MRI system of the present disclosure.

FIG. 6 is a flowchart setting forth some non-limiting example steps of a method for using the portable MRI system in accordance with some aspects of the present disclosure.

FIG. 7 is a pulse sequence diagraph of a diffusion pulse sequence for use with the portable MRI system of the present disclosure.

FIG. 8 is a set of reference frames illustrating a method for orthogonal diffusion encoding using the portable MRI system in accordance with some aspect of the present disclosure.

FIG. 9 shows example images with and without static and dynamic correction of electromagnetic interference in accordance with the present disclosure.

FIG. 10 is a graphical representation of a method to correct for electromagnetic interference in accordance with the present disclosure.

FIG. 11 is a block diagram of an example magnetic resonance imaging (“MRI”) system that can implement the methods described in the present disclosure.

FIG. 12 is a block diagram of an example MRI system that can implement the methods of the present disclosure.

FIG. 13 is a block diagram of example components that can implement the system of FIG. 12.

### DETAILED DESCRIPTION

Before any aspects of the disclosure are explained in detail, it is to be understood that the invention is not limited in its application to the details of construction and the arrangement of components set forth in the following description or illustrated in the following drawings. The invention may be realized in any of a variety of configurations and may be practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

“Homogeneous” or “nearly homogeneous” are used to describe a field within a generally-acceptable range. In some situations, that may be ±2% of the mean value within the volume or region of interest, and in others it might be greater, such as ±20%. For example, traditional MRI requires greater homogeneity, whereas some techniques, such as magnetic resonance fingerprinting, needs much less homogeneity. Regarding diffusion weighted imaging (DWI), in practice, acquiring an image with b=0 s/mm² will have a small diffusion weighting applied. Thus, “b≈0 s/mm²” and “b=0 s/mm²” can be understood as b<30 s/mm². “Approximately orthogonal” may be defined as 90°±10°.

The following discussion is presented to enable a person skilled in the art to make and use aspects of the invention. Various modifications to the illustrated embodiments will be readily apparent to those skilled in the art, and the generic principles herein can be applied to other embodiments and applications without departing from the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

The lack of accessibility of MRI to NICU patients is a problem that has garnered attention and innovation in the MR community. Available MR-compatible incubators can address some of the safety concerns related to transfer, temperature stability, and monitoring. However, it is not realistic to replace all NICU incubators with these specialized, costly, MR compatible incubators. Therefore, currently, the only feasible workflow still requires transfer of the patient into the MR compatible incubator for transport to the radiology facility. Thus, this approach unfortunately encompasses many of the downsides of standard MRI transfer, i.e., disruption to care equipment, associated stress to the patient, and staffing concerns.

To avoid exiting the NICU, there has been work on specialized neonatal scanners that are sited in the NICU. These systems are based on extremity scanners that were modified for the neonatal application. The small footprint of these scanners allows them to be sited in a smaller MR suite within the NICU, but they still require a stationary radiofrequency (RF) shielded room and ferrous exclusion zone. The Embrace scanner from Aspect Imaging is a 1 T permanent magnet scanner that was specifically designed for the NICU and incorporates a small, specialized incubator, monitoring, and video surveillance equipment. This scanner also has the advantage of not requiring a shielded room, but its sizable magnetic footprint, weight, and size preclude portability for true bedside use.

While these dedicated NICU scanners are a step in the right direction, they are installed in a fixed location, and therefore still require transport of the patient across the NICU to the scanner. This still generates non-negligible disruption to the care conditions. Realistically, these types of scanners will only be installed in very large NICU units with an abundance of resources. Given the size of these units, the substantial transport distance and transfer of the patient to the scanner could take 30 minutes or more and require a 3-4-person care team and the removal and replacement of medical monitoring equipment. In the end, the transfer to the stationary NICU scanner may only be modestly more convenient than transfer to a conventional 3 T scanner, which provides higher image quality.

Although not designed for pediatric applications, some have created portable brain scanners. These scanners have the major advantage of being transportable to the patient's bedside and their low field and head-only design may allow use of standard monitoring and support equipment. However, the smaller size of the neonatal brain and other shortcomings stop such systems from being applicable to this patient base. For example, stringent image resolution requirements for neonatal applications undermine the opportunity to use such generalized, portable head or other portable MRI system from being used in neonatal applications. Further, the NICU presents a workflow environment that is unique. For example, because many patients are arranged in close proximity and with many critical monitoring systems in use, the industrial design of any system introduced into the NICU must minimize disruption to the patient's care, not interfere with the care of others in the NICU or the systems monitoring those in the NICU, and be sufficiently compact and maneuverable to be able to physically traverse a busy NICU.

The system and methods described herein establish a specialized solution for cranial imaging, which offers the portability, safety, silence, and cost of ultrasound (US) as well as the diagnostic information of MRI. The scanner provides early, additional, and useful diagnostic information beyond the capabilities of US. The system can include “push-button” operation that can reduce or eliminate user variability and reduce the dependence on specialized personnel, a well-known disadvantage of US.

In one non-limiting example, for neonates exhibiting abnormal neurological function, the workflow can include, 1) transporting the MRI scanner cart to the patient's bedside, 2) lateral transfer (<60 cm) of the infant to the scanner bed, and 3) a series of 5-10 minute push-button, near-silent scans with multiple contrast options, such as T₁-weighted, T₂-weighted, and DWI. The short transfer distance allows supportive care and monitoring equipment to remain in place and, thus, eliminates the need to remove and reapply necessary tubes, leads, IVs, or the like.

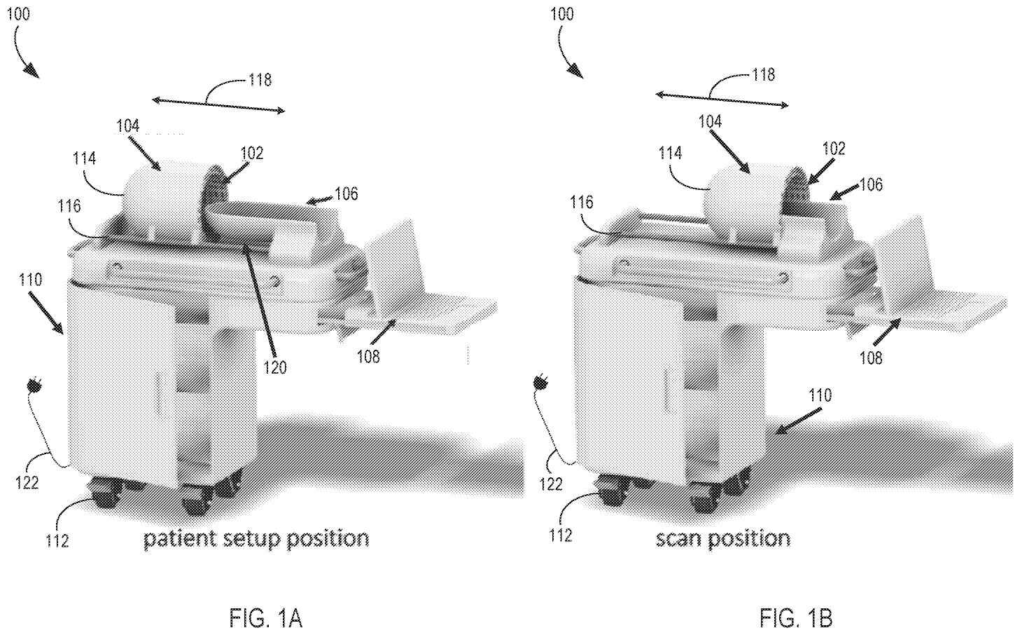

FIGS. 1A and 1B illustrate an example of some aspects of a portable neonatal MRI system 100 according to the present disclosure. The portable neonatal MRI system 100 can include a static field (B₀) magnet 102, gradient coils 104, a patient bed 106, and a console control 108. The system can be lightweight and relatively small, allowing it to be placed on a movable cart 110 to be moved directly to the patient's incubator/bedside. For example, the cart 110 may be fashioned with wheels 112 to be pushed by medical personnel or may be otherwise movable, such as along a track or by a vehicle. The cart 110 may have a small footprint for easy navigation around the NICU and facilitate short (e.g., <60 cm) transfer of the infant to the patient bed 106. The short transfer distance will allow supportive care and monitoring equipment to remain in place, eliminating the need to remove and reapply tubes, leads, IVs, etc. The cart 110 may also have a variable height to be height-matched to the patient's bedside, allowing for easy transfer. The system 100 may also comprise a power supply 122. In some configurations, the power supply 122 may be a standard (e.g., 120 V and 15 A) power outlet to facilitate use in any NICU without the need for specialized power equipment.

To realize this design, the gradient coils 104 may be placed inside or outside the B₀ magnet 102 to enable the patient to be closer to the B₀ magnet 102, which maximizes the field efficiency. The B₀ magnet 102 and gradient coils 104 may be configured together into one helmet-like housing 114, such that a newborn can be slid in and out of the housing 114. The housing 114 may be movable relative to the patient bed 106. For example, the housing 114 and patient bed 106 may be fashioned on a track 116 that allows for motion in the direction along the axis 118. In this way, the patient may be placed in the patient bed 106 while the system 100 is in the position indicated in FIG. 1A. Then, the housing 114 may be slid towards the patient bed 106 to the position shown in FIG. 1B for scanning. Alternatively, the patient bed 106 can be slid towards the housing 114 to realize the relative positions shown in FIG. 1B. Additionally or alternatively, the B₀ magnet 102 and gradient coils 104 may be separably movable relative to the patient bed 106. The patient bed 106, housing 114, B₀ magnet 102, and gradient coils 104 may be otherwise movable for easy placement of the patient into a position for scanning. For example, the patient bed 106 or housing 114 may be movable on wheels, by a motor, by an operator, by an automated system controlled by the console control 108, etc. There may be a space 120 between the patient bed 106 and the top surface of the cart 110, such that the housing 114 may fully surround the head of the patient when in the scanning position to provide a homogeneous B₀ field.

The B₀ magnet 102 and gradient coils 104 may alternatively be configured in two separate housings for increased flexibility, if desired. For example, the system may comprise more than one B₀ magnets 102 of varying sizes, which may be interchangeably placed over the patient depending on the patient's size. This may allow a close fit for maximized magnetic flux in the scanning volume for smaller patients.

The console control 108 may be placed on the cart 110 as shown in FIGS. 1A and 1B or may be placed otherwise nearby the cart 110. The console control 108 may be used to control image acquisition. For example, the console control 108 may be configured for push-button scanning of the patient. The console control 108 may also be used to select imaging parameters, such as field of view (FOV), resolution, contrast, repetition time (TR), echo time (TE), etc. The console control 108 may also be configured to display the acquired images or other patient records for quality control, fast image interpretation, etc. The console control 108 may also be in wireless or other communication with a network or server to send or receive data, such as imaging data, patient data, reconstructed images, etc.

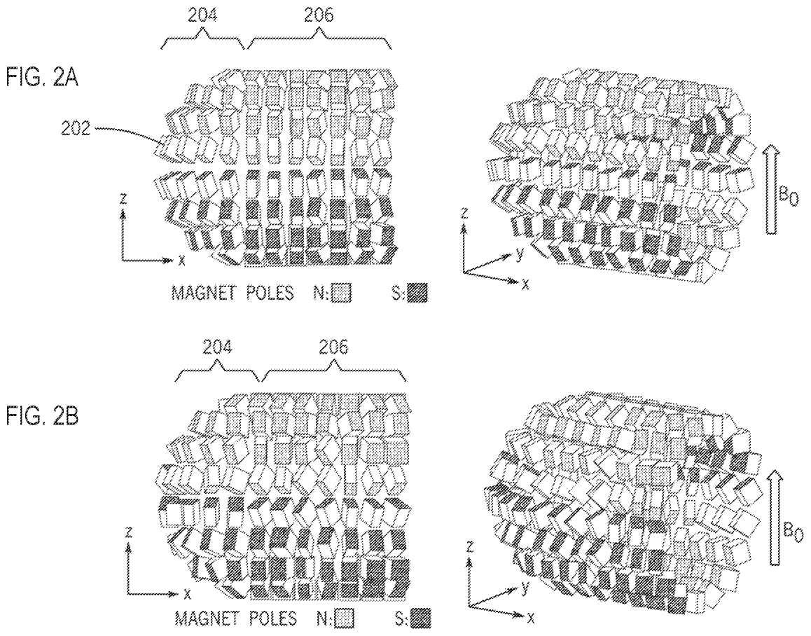

The B₀ magnet 102 is further illustrated in the non-limiting examples shown in FIGS. 2A and 2B. The B₀ magnet 102 may be a low-field permanent magnet comprising several magnetic elements 202. The magnetic elements 202 may, optionally, be permanent magnets. Further, the magnetic elements 202 may be arranged in a Halbach array. In one non-limiting example, the Halbach array may be arranged in a Halbach bulb configuration, which merges a Halbach cylinder and Halbach sphere to conform around the head and produce an efficient field-to-weight ratio. Alternatively, only a Halbach cylinder or only a Halbach sphere may be used. Thus, as used herein, Halbach array may be used to refer to any of a variety of shapes, including a Halbach cylinder, a Halbach sphere, or a combination of these or other shapes, for example, such as a Halbach bulb. However, as a non-limiting example, the Halbach bulb will be described, for example, because it includes both the Halbach cylinder and the sphere.

The Halbach bulb is characterized by two ends. The first end 204 is constructed as a partial Halbach sphere. The second end 206 is constructed as a Halbach cylinder. The cylindrical end is open such that a patient can be placed into the coil by moving the patient in a direction parallel to the axis of the cylinder. Combining the Halbach sphere with the Halbach cylinder produces higher efficiency per unit mass of permanent magnetic elements than the Halbach cylinder alone.

The number, size, position, and orientation of the permanent magnetic elements 202 may be optimized using simulation for a desired B₀ field over a target scanning volume and constrained by weight and size requirements. For example, the B₀ magnet 102 may be designed to create a main field within the range of 0.01 T to 1 T. The Halbach array may be arranged to achieve the desired magnetic field over a desired scanning volume. For example, the Halbach array may be designed to produce a maximally homogeneous field over an 8-20 cm sphere to facilitate scanning of the neonatal brain. The scanning region may also be chosen to be another volume based on size or anatomy of the target patient population. The B₀ magnet 102 may be lightweight and small for portability and ease of use. For example, the B₀ magnet may weigh less than a target weight of <15 kg, <25 kg, <50 kg, etc. and have a size of <30×30×30 cm³, <50×50×50 cm³, etc. The permanent magnetic elements 202 may be constructed from magnetic blocks, such as NdFeB or Samarium-Cobalt (SmCo or Sm(Co,Fe,CuZr)₇) blocks. The blocks may be of various strengths and sizes.

In addition to using an optimization process to achieve a homogeneous B₀ field, the system 100 may optionally include other ferromagnetic materials for passive shimming. The placement of these passive shims may be optimized using a standard B₀ field mapping technique prior to patient scanning. The system may also include one or more field probes that can statically or dynamically measure B₀ field inhomogeneity for retrospective correction by standard approaches or other methods known in the art.

The array of the B₀ magnet 102 may further be designed to include a built-in gradient for read-out encoding. The built-in gradient can reduce or eliminate the need for the standard read-out gradient system, which can advantageously reduce acoustic noise, power requirements, heating, system size, and cost. For example, the magnetic elements in the Halbach array may be oriented to produce a main magnetic field with a linear gradient of 1-50 mT/m along an axis of the scanning volume. As one non-limiting example, the arrangement shown in FIG. 2B achieves a 10 mT/m gradient with a linearity within 20% along the x-axis. In an arrangement that includes a field gradient (e.g., FIG. 2B), scanning sequences may be limited to spin-echoes, such as rapid imaging with refocused echoes (RARE), which still allows standard T₂, inversion-recovery (IR) prepped T₂, T₁, proton density (PD), and diffusion contrasts.

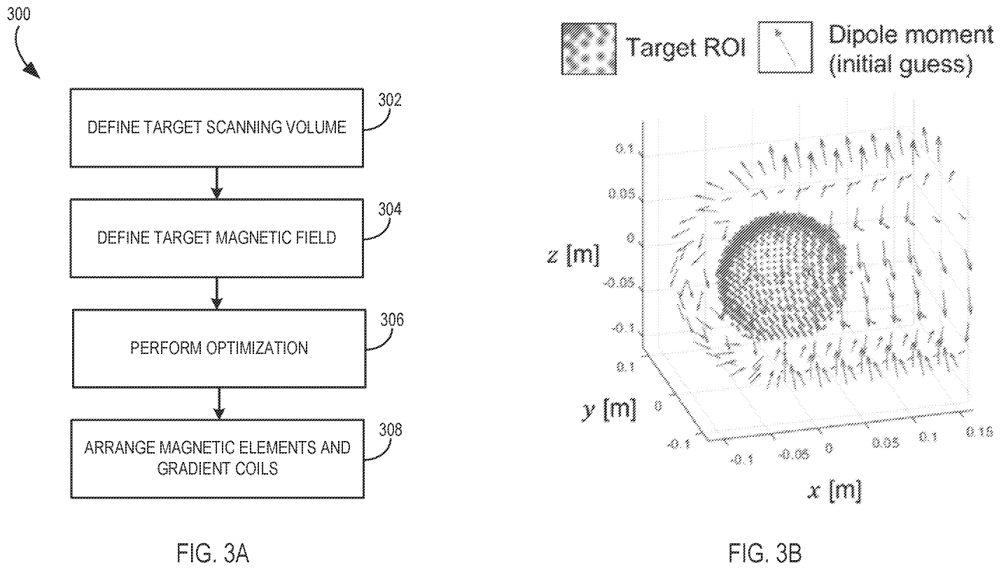

Referring now to FIG. 3A, a process 300 for designing a neonatal MRI system is illustrated. The Halbach array arrangement can be optimized to generate a desired magnetic field as defined in block 304 within a target scanning volume as defined in block 302. For example, the desired target magnetic field can be a homogeneous static magnetic field (B₀) at a desired field strength, such as 0.1 T. The target magnetic field may also be defined as a functionally linear field such that the functionally linear field is capable of standard spatial encoding to acquire k-space data. The target scanning volume can be defined based on the imaging application. For example, for neonatal brain imaging, the target volume may be defined as 10-20 cm sphere at the center of the Halbach bulb. To optimize the arrangement of the permanent magnetic elements 202, a genetic algorithm may be used. An optimization may be used in block 306 to determine the size, position, or orientation of each of the permanent magnetic elements 202 within the Halbach array. It may also determine the total number of elements within the array. For practicality and portability, the optimization of block 306 can be constrained by the total size or total weight of the array. The algorithm may minimize the absolute range of the simulated B₀ magnitude of the target imaging volume or minimize variation from the desired gradient or other field pattern. The optimization may be constrained to maintain a mean B₀ at a desired field strength. The optimization may also constrain the magnetic dipole moment magnitude of each block to not exceed that of a 1″×1″×1″ block of N52 magnet material or other sized block of magnetic material. The size of the block can be varied within the optimization. Each magnet block may be modeled using a subset of multipole components (e.g., first, third, and fifth). The algorithm can determine a desired magnetic moment for each block, as illustrated in FIG. 3B, for example. The magnetic moments can then be converted into a corresponding block volume and used to generate a design of discrete permanent magnet blocks in space. The generated design may be further validated using BiotSavart simulations, which can be configured to consider multipole terms of up to fifth order, for example, or higher. The output design can provide an orientation, size, placement, and total number of the permanent magnetic elements 202, which can be arranged in the optimal arrangement in block 308.

As one non-limiting example, as shown in FIGS. 2A and 2B, an optimized array of 184 NdFeB blocks of grade N52 may be arranged in a Halbach bulb to produce a nearly homogeneous (as defined by the situation or application) main magnetic field of 125 mT over a 14 cm spherical scanning volume. The array may weigh less than 15 kg and have the maximum dimensions of 30 cm, 25 cm, and 25 cm, along x, y, and z respectively. According to BiotSavart simulations, the design illustrated in FIG. 2A achieved a B₀ field of 127.2 mT±1.73 mT over the 14 cm spherical imaging volume. The design shown in FIG. 2B can achieve a mean B₀ field of 126.7 mT and a 10 mT/m gradient with an error of up to 3 mT over the imaging volume.

The gradient coils 104 may be used for phase encoding in the y and z directions, for example. The gradient coils 104 can be positioned on the outer surface of the B₀ magnet 102. This positioning reduces acoustic noise by placing the permanent magnet material between the noise source and the infant and positioning the coils in a relatively low-field region (i.e., outside of the magnet). The strategy also provides a smaller inner magnet diameter to maximize field strength. This positioning is typically not possible for conventional MRIs and is made possible by the minimal eddy current effects of the permanent magnetic elements 202 (e.g., NdFEB).

The switchable phase encoding gradients may operate at low-power levels without the need for cooling, especially when the scanning sequences are limited to spin-echo sequences. In spin-echo sequences, the B₀ inhomogeneity is refocused in the spin-echo. Thus, the phase encoding gradients are not required to dominate in the pulse sequence. Efficiency of the gradient coils 104 may be optimized using small coil sizes and by reducing or eliminating the need of a shielding layer using a sparse permanent magnet. The gradient coils 104 may require a peak power <10 A for the imaging sequences and operate with passive air-cooling. The efficient design may also allow for the integration of low-cost, low-power, small-footprint operational amplifier-based drivers. Alternatively, the gradients may be driven by larger gradient drivers, such as AE Techron 7224 amplifiers. The system 100 may further include a fan for forced air cooling or other cooling system. Further, the system 100 may include an optional field probe monitor to correct for heating effects of the permanent magnets in post processing steps.

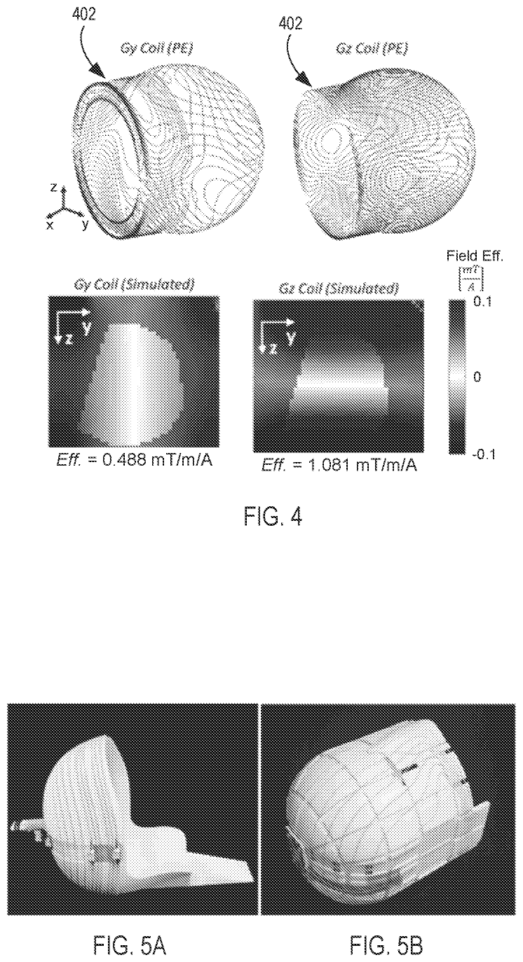

Referring to FIG. 4, the gradient coils 104 may be designed to create a linear target field along the imaging volume in the y and z directions, for example. The current paths may be optimized using a stream function boundary element method (SF-BEM) solver. An example of gradient coil paths for integration with a Halbach bulb B₀ magnet is shown in FIG. 4. For example, the coil winding patterns 402 may be designed for a target gradient efficiency of 1 mT/m/A and a DC resistance of <2 ohms. The coil winding patterns 402 may be placed on the inner and outer surfaces of a magnet former, which may be fit outside the permanent magnet. As a non-limiting example, the former may be 3D printed with winding pattern grooves to accommodate wire (e.g., AWG 18 wire).

They system 100 may also include transmit (Tx), receive (Rx), or transmit/receive (Tx/Rx) radiofrequency (RF) coils. As a non-limiting example, a joint Tx/Rx coil can be used to save space. The Tx/Rx coil may be a single channel helmet coil with spiral geometry, as shown in FIGS. 5A and 5B. Examples of a spiral coil are described in US Patent Publication 2017/0003359 or International Publication WO2021217137. Advantageously, single channel spiral receive coil helmets can be highly sensitive at low fields. Specific absorption rate (SAR) is also not an issue at low fields (e.g., 4.3 MHZ). However, in a head-only scanner, a uniform spiral winding may cause 1) B₁″ inhomogeneity and flip-angle variation and 2) a B₁ field that extends outside the linear gradient region, which may result in signal acquisition in the neck region that can alias into the imaging FOV. Thus, the coil design may comprise a spiral coil with a variable turn density, compared to uniform windings, as shown in FIG. 5A. The example design in FIG. 5A reduces the B₁⁺ inhomogeneity by 7-9% in the imaging volume, compared to uniform windings. The coil may also be designed with a target field, stream function approach to produce a transverse field with reduced coil sensitivity in the neck, as done in FIG. 5B, for example. The winding pattern may be further optimized using the BEM-SF method. The optimized pattern may be 3D printed as grooves in close-fitting helmets, as shown in FIGS. 5A and 5B. The coils may be tuned and matched with a low-Q (e.g., <100) to conform with the bandwidth of the built-in permanent encoding field.

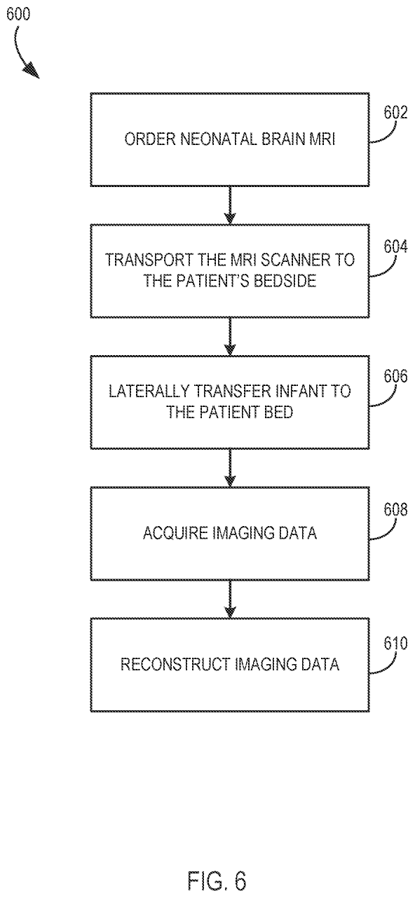

Referring now to FIG. 6, an example process 600 is presented for point-of-care neonatal imaging. In process block 602 a brain MRI may be ordered by a medical professional for a neonate. For example, the patient may be in the NICU and have suspected HIE, meningitis, stroke, or other clinical condition. The point-of-care system 100 may be transported to the patient's bedside in block 604, and the patient can be laterally transferred (e.g., <60 cm) to the patient bed 106 of the scanner in block 606. For example, transferring the patient in block 606 may advantageously not require the removal of life-saving medical equipment (e.g., feeding tubes, IVs, etc.) from the patient. Imaging data can be acquired in block 608 by controlling the scanner using the console control 108. In block 610 the imaging data can be processed by the console control 108 or by an external server to generate anatomical images, quantitative maps, or other data.

Acquiring imaging data in block 608 may include an imaging protocol of various sequence types. For example, the acquisition may be based on 3D RARE sequences utilizing the built-in readout gradient. The sequence may use a frequency-swept RF excitation and refocusing pulses due to the wide Larmor frequency bandwidth. Partition phase encoding may be performed along the echo train, and in-plane phase encoding may be performed shot-to-shot.

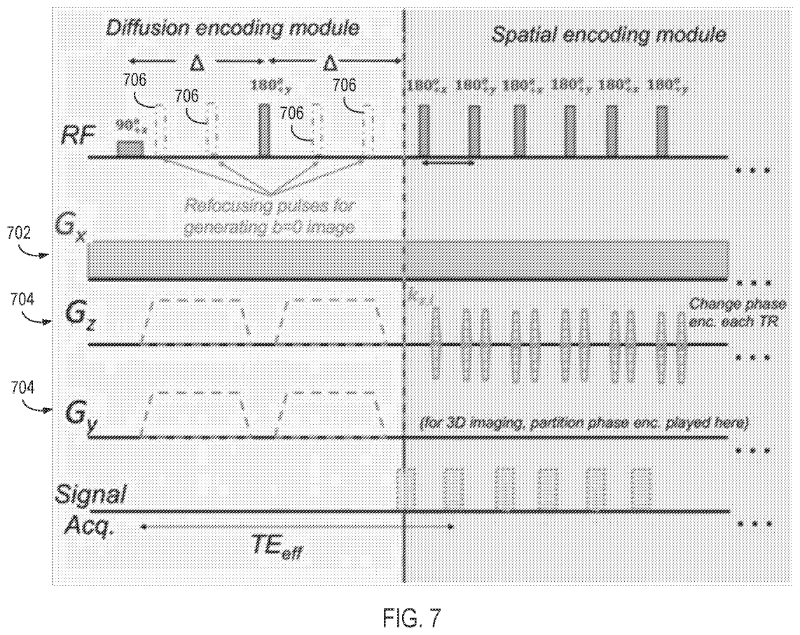

A diffusion weighted imaging (DWI) sequence may also be acquired, which may be required to fully characterized the HIE state. To achieve DWI in an inhomogeneous field, the sequence shown in FIG. 7 may be used. The DWI sequence may be based on an extension of the Carr-Purcell RARE sequence, in which most of the diffusion encoding occurs in the time-period before the first echo. The permanent gradient 702 can be used for readout encoding along one direction (e.g., x). Switchable gradients 704 (e.g., along z or y) can be used to produce phase encoding in one or two dimensions for 2D or 3D imaging, respectively.

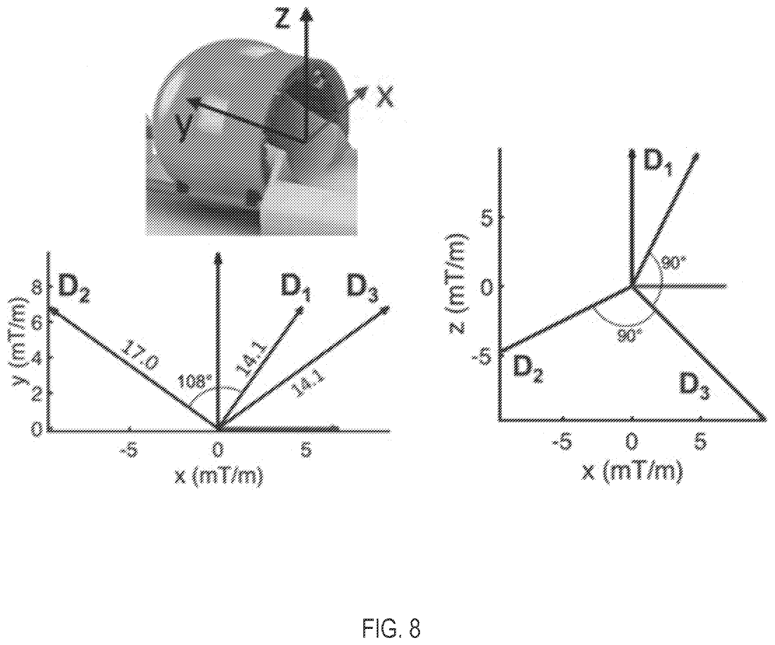

In order to produce images with negligible diffusion weighting (e.g., b<30 s/mm²) in the presence of a permanent gradient, the DWI pulse sequence may include dummy refocusing pulses 706 that can be used to effectively turn off diffusion encoding. The permanent gradient 702 can be used to create diffusion encoding along one dimension. For example, if the permanent gradient is linear along x, the diffusion encoding will provide information about the diffusion along x. Diffusion encoding can also be applied in three approximately orthogonal directions using the permanent gradient along with two or more switchable gradients 704. For example, the switchable gradients 704 can be intermittently applied by switchable gradient coils along the y and z directions using the gradient coils 104. The permanent gradient 702 can be continually applied along the x direction. As a non-limiting example, using the combination of gradients shown in FIG. 7 can produce the diffusion encoding along the directions illustrated in FIG. 8. Apparent diffusion coefficient images can be produced after acquiring images with at least two different b-values, such as b≈0 s/mm² and b=800 s/mm² along one or more directions.

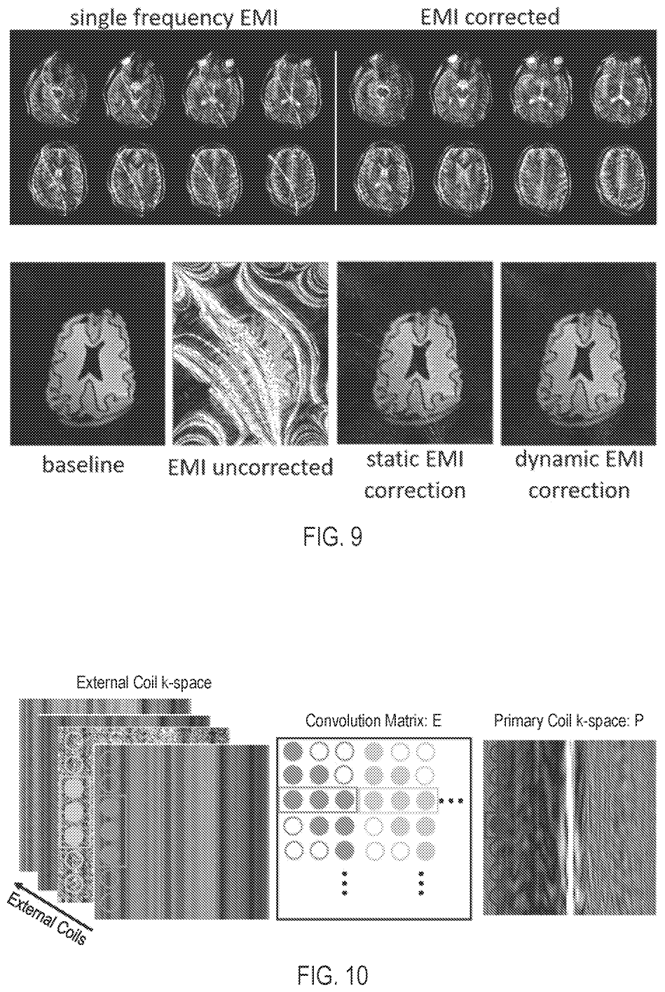

Referring now to FIGS. 9 and 10, a strategy to compensate for ambient electromagnetic interference (EMI) is presented. The open geometry bedside system allows for the removal of the traditional RF shielding around the MRI suite. A passive copper shield may be included between the B₀ magnet and RF coil. The passive shield can reduce EMI significantly. However, given the stringent SNR demands of neonatal imaging and the likelihood of additional sources of EMI during bedside imaging, residual EMI may still negatively affect image quality. Thus, additional EMI correction may be used to retrospectively attenuate the effects of ambient or environmental EMI. In particular, one or more external coils can be mounted to the scanner cart or otherwise placed in or around the system. These external RF coils can be tuned to measure environmental electromagnetic interference from various sources. The external RF coils can be configured to measure RF electromagnetic signal simultaneously as the primary imaging RF coil measures imaging data from the patient.

To retrospectively remove the external EMI signal, a transfer function can be calculated that relates the imaging RF coil data with the external RF coil data. For example, the transfer function can approximate the correction as a static single frequency EMI source based on a single external coil. As a non-limiting example, the in vivo brain images shown in FIG. 9 were acquired in the presence of a 3.38 MHz EMI signal resulting in the original images. The corrected images are shown, after removing the single-frequency signal from the imaging data.

The approach can be further generalized to model realistic EMI sources. For example, assuming N external RF coils, the signal measured can be represented as Eᵢ(kₓ, k_{y}), which is related to the EMI observed by the primary coil according to:

$$S ( {k}_{x} , {k}_{y} ) = {Σ}_{i = 1}^{N} {E}_{i} ( {k}_{x} , {k}_{y} ) * {H}_{i} ( {k}_{x} , {k}_{y} )$$

where Hᵢ(kₓ, k_{y}) represents the impulse response function for the i^{th} external RF coil. A(Kₓ, K_{y}) limited support window can be assumed for each response function, i.e., Hᵢ(kₓ, k_{y})=0, |kₓ|>0 or k_{y}|>0.

The primary imaging coil will observe the combination of the EMI and the true image (I) according to: P(kₓ, k_{y})=S(kₓ, k_{y})+I(kₓ, k_{y}). Thus, the transfer function can be found by solving a linear system: H=E†P. As the true image and EMI spectrum are unlikely to be correlated, the estimated primary coil EMI (S≈EH) can be directly removed from the primary coil data (P).

This method can be extended to account for time-varying EMI by binning each phase encoding line so that time-localized transfer functions can be fit. FIG. 10 demonstrates the application of a time-varying transfer function used to model EMI that was recorded by four external RF coils. In this non-limiting example, a narrow window of (Kₓ, K_{y})=(5,1) was assumed and transfer functions were fit for each individual phase encoded line. The phase encoded lines were then grouped based upon correlation of transfer functions, which represents similarity of EMI sources over time. This automated grouping can facilitate the computation of a compact set of transfer functions to represent the time-varying EMI.

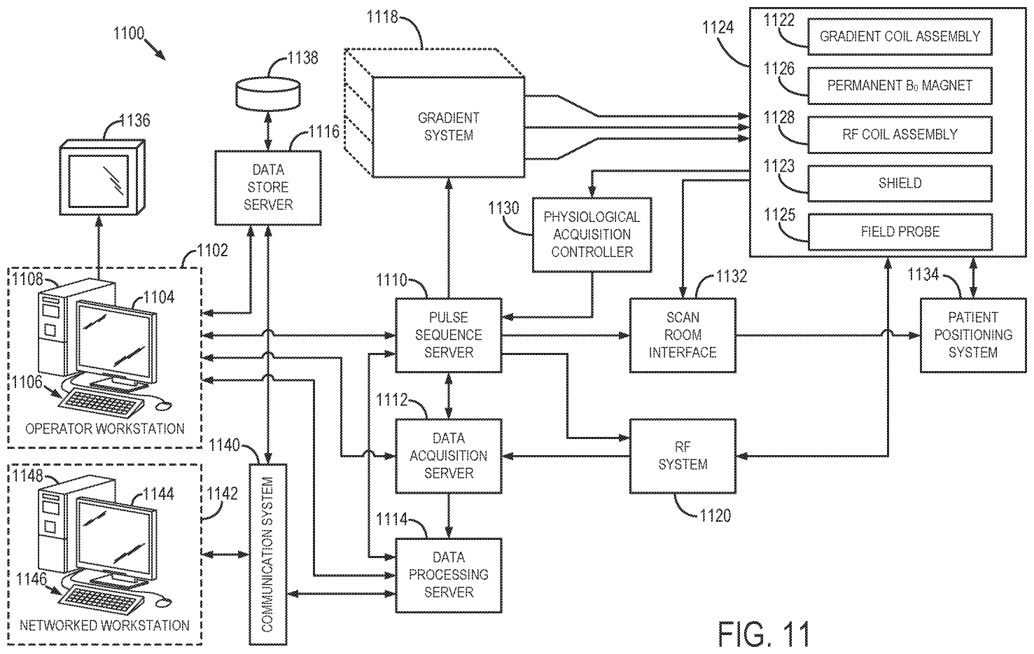

Referring particularly now to FIG. 11, an example of some embodiments of a portable neonatal MRI system 1100 is illustrated. The MRI system 1100 includes an operator workstation 1102 that may include a display 1104, one or more input devices 1106 (e.g., a keyboard, a mouse), and a processor 1108 that collectively control data acquisition of the MRI system 1100. The processor 1108 may include a commercially available programmable machine running a commercially available operating system. The operator workstation 1102 provides an operator interface that facilitates entering scan parameters into the MRI system 1100. The operator workstation 1102 may be coupled to different servers, including, for example, a pulse sequence server 1110, a data acquisition server 1112, a data processing server 1114, and a data store server 1116. The operator workstation 1102 and the servers 1110, 1112, 1114, and 1116 may be connected via a communication system 1140, which may include wired or wireless network connections.

The pulse sequence server 1110 functions in response to instructions provided by the operator workstation 1102 to operate a gradient system 1118 and a radiofrequency (“RF”) system 1120. Gradient waveforms for performing a prescribed scan are produced and applied to the gradient system 1118, which then excites gradient coils in an assembly 1122 to produce the magnetic field gradients (e.g., Gₓ, G_{y}, or G_{z}) that can be used for spatially encoding magnetic resonance signals, with or without the presence of a permanent gradient. The gradient coil assembly 1122 forms part of a magnet assembly 1124 that includes a gradient coil assembly 1122, a permanent B₀ magnet 1126, an RF coil assembly 1128, a shield 1123, and a field probe 1125. For example, the shield 1123 may be a passive copper shield that is positioned between the B₀ permanent magnet 1126 and the imaging RF coil. The shield 1123 may be configured to reduce EMI. The RF coil assembly 1128 may include an imaging coil and an externally mounted coil tuned to measure environmental EMI interference. The one or more field probes 1125 may be configured to measure B₀ inhomogeneity.

RF waveforms are applied by the RF system 1120 to the RF coil assembly 1128, or a separate local coil to perform the prescribed magnetic resonance pulse sequence. Responsive magnetic resonance signals detected by the RF coil assembly 1128, or a separate local coil, are received by the RF system 1120. The responsive magnetic resonance signals may be amplified, demodulated, filtered, and digitized under direction of commands produced by the pulse sequence server 1110. The RF system 1120 includes an RF transmitter for producing a wide variety of RF pulses used in MRI pulse sequences. The RF transmitter is responsive to the prescribed scan and direction from the pulse sequence server 1110 to produce RF pulses of the desired frequency, phase, and pulse amplitude waveform. The generated RF pulses may be applied to the RF coil assembly 1128 or to one or more local coils or coil arrays.

The RF system 1120 also includes one or more RF receiver channels. An RF receiver channel includes an RF preamplifier that amplifies the magnetic resonance signal received by the RF coil assembly 1128 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}}$$

and the phase of the received magnetic resonance signal may also be determined according to the following relationship:

$$φ = {tan}^{- 1} ( \frac{Q}{I} ) .$$

The pulse sequence server 1110 may receive patient data from a physiological acquisition controller 1130. By way of example, the physiological acquisition controller 1130 may receive signals from a number of different sensors connected to the patient, including electrocardiogramals from electrodes, or respiratory signals from a respiratory bellows or other respiratory monitoring devices. These signals may be used by the pulse sequence server 1110 to synchronize, or “gate,” the performance of the scan with the subject's heartbeat or respiration.

The pulse sequence server 1110 may also connect to a scan room interface circuit 1132 that receives signals from various sensors associated with the condition of the patient and the magnet system. Through the scan room interface circuit 1132, a patient positioning system 1134 can receive commands to move the patient to desired positions during the scan.

The digitized magnetic resonance signal samples produced by the RF system 1120 are received by the data acquisition server 1112. The data acquisition server 1112 operates in response to instructions downloaded from the operator workstation 1102 to receive the real-time magnetic resonance data and provide buffer storage, so that data are not lost by data overrun. In some scans, the data acquisition server 1112 passes the acquired magnetic resonance data to the data processor server 1114. In scans that require information derived from acquired magnetic resonance data to control the further performance of the scan, the data acquisition server 1112 may be programmed to produce such information and convey it to the pulse sequence server 1110. For example, during pre-scans, magnetic resonance data may be acquired and used to calibrate the pulse sequence performed by the pulse sequence server 1110. As another example, navigator signals may be acquired and used to adjust the operating parameters of the RF system 1120 or the gradient system 1118, or to control the view order in which k-space is sampled. In still another example, the data acquisition server 1112 may also process magnetic resonance signals used to detect the arrival of a contrast agent in a magnetic resonance angiography (“MRA”) scan. For example, the data acquisition server 1112 may acquire magnetic resonance data and processes it in real-time to produce information that is used to control the scan.

The data processing server 1114 receives magnetic resonance data from the data acquisition server 1112 and processes the magnetic resonance data in accordance with instructions provided by the operator workstation 1102. Such processing may include, for example, reconstructing two-dimensional or three-dimensional images by performing a Fourier transformation of raw k-space data, performing other image reconstruction algorithms (e.g., iterative or back projection reconstruction algorithms), applying filters to raw k-space data or to reconstructed images, generating functional magnetic resonance images, or calculating motion or flow images.

Images reconstructed by the data processing server 1114 are conveyed back to the operator workstation 1102 for storage. Real-time images may be stored in a data base memory cache, from which they may be output to operator workstation 1102 or a display 1136. Batch mode images or selected real time images may be stored in a host database on disc storage 1138. When such images have been reconstructed and transferred to storage, the data processing server 1114 may notify the data store server 1116 on the operator workstation 1102. The operator workstation 1102 may be used by an operator to archive the images, produce films, or send the images via a network to other facilities.

The MRI system 1100 may also include one or more networked workstations 1142. For example, a networked workstation 1142 may include a display 1144, one or more input devices 1146 (e.g., a keyboard, a mouse), and a processor 1148. The networked workstation 1142 may be located within the same facility as the operator workstation 1102, or in a different facility, such as a different healthcare institution or clinic.

The networked workstation 1142 may gain remote access to the data processing server 1114 or data store server 1116 via the communication system 1140. Accordingly, multiple networked workstations 1142 may have access to the data processing server 1114 and the data store server 1116. In this manner, magnetic resonance data, reconstructed images, or other data may be exchanged between the data processing server 1114 or the data store server 1116 and the networked workstations 1142, such that the data or images may be remotely processed by a networked workstation 1142.

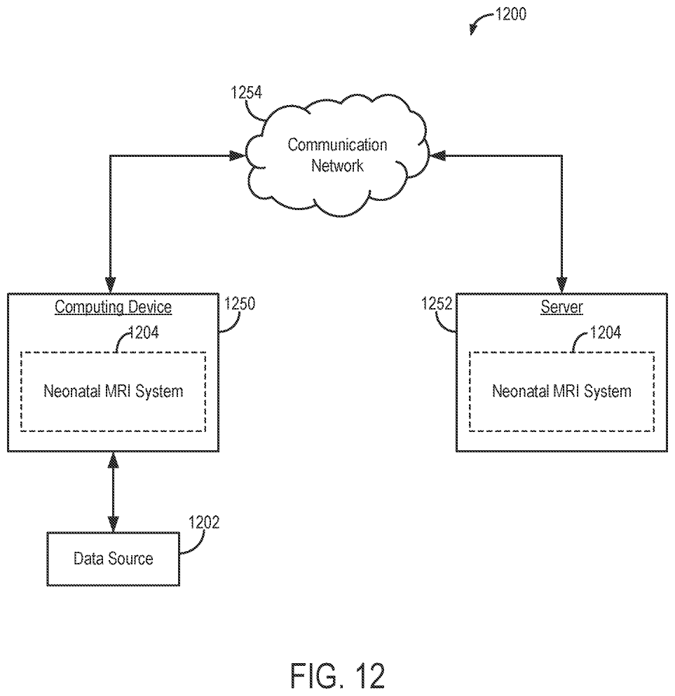

Referring now to FIG. 12, an example of a system 1200 for implementing neonatal MRI methods from data acquired using a portable neonatal MRI system in accordance with some aspects of the systems and methods described in the present disclosure is shown. As shown in FIG. 12, a computing device 1250 can receive one or more types of data (e.g., signal evolution data, k-space data, receiver coil sensitivity data) from data source 1202. In some configurations, computing device 1250 can execute at least a portion of a neonatal MRI system 1204 to reconstruct images from magnetic resonance data (e.g., k-space data) acquired using a reconstruction technique. In some configurations, the neonatal MRI system 1204 can implement an automated pipeline (e.g., 100) to provide images.

Additionally or alternatively, in some configurations, the computing device 1250 can communicate information about data received from the data source 1202 to a server 1252 over a communication network 1254, which can execute at least a portion of the neonatal MRI system 1204. In such configurations, the server 1252 can return information to the computing device 1250 (and/or any other suitable computing device) indicative of an output of the neonatal MRI system 1204.

In some configurations, computing device 1250 and/or server 1252 can be any suitable computing device or combination of devices, such as a desktop computer, a laptop computer, a smartphone, a tablet computer, a wearable computer, a server computer, a virtual machine being executed by a physical computing device, and so on. The computing device 1250 and/or server 1252 can also reconstruct images from the data.

In some configurations, data source 1202 can be any suitable source of data (e.g., measurement data, images reconstructed from measurement data, processed image data), such as an MRI system, another computing device (e.g., a server storing measurement data, images reconstructed from measurement data, processed image data), and so on. In some configurations, data source 1202 can be local to computing device 1250. For example, data source 1202 can be incorporated with computing device 1250 (e.g., computing device 1250 can be configured as part of a device for measuring, recording, estimating, acquiring, or otherwise collecting or storing data). As another example, data source 1202 can be connected to computing device 1250 by a cable, a direct wireless link, and so on. Additionally or alternatively, in some configurations, data source 1202 can be located locally and/or remotely from computing device 1250, and can communicate data to computing device 1250 (and/or server 1252) via a communication network (e.g., communication network 1254).

In some configurations, communication network 1254 can be any suitable communication network or combination of communication networks. For example, communication network 1254 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 1254 can be a local area network, a wide area network, a public network (e.g., the Internet), a private or semi-private network (e.g., a corporate or university intranet), any other suitable type of network, or any suitable combination of networks. Communications links shown in FIG. 12 can each be any suitable communications link or combination of communications links, such as wired links, fiber optic links, Wi-Fi links, Bluetooth links, cellular links, and so on.

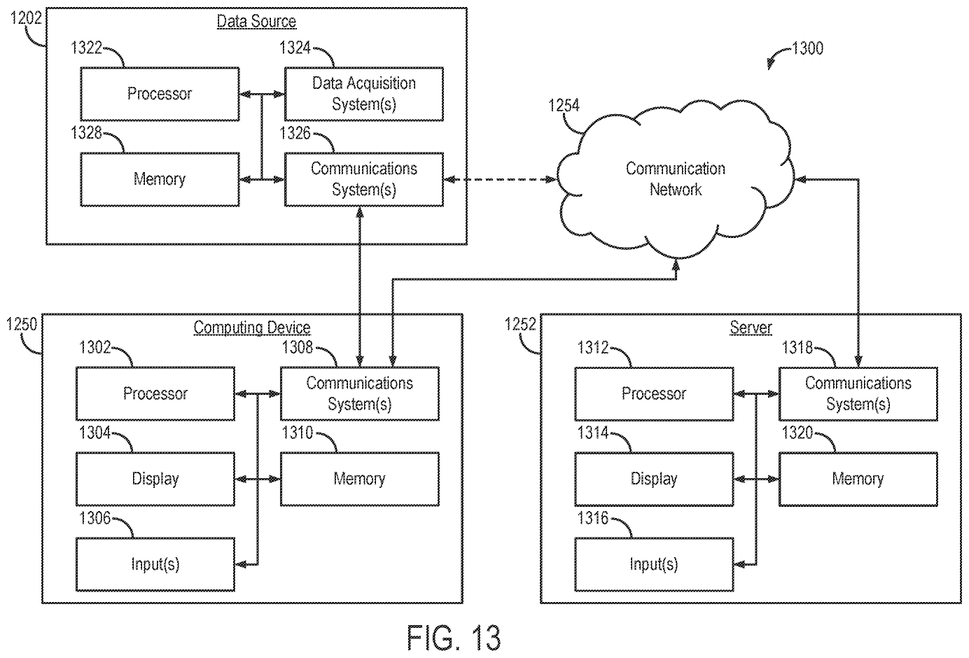

Referring now to FIG. 13, an example of hardware 1300 that can be used to implement data source 1202, computing device 1250, and server 1252 in accordance with some configurations of the systems and methods described in the present disclosure is shown.

As shown in FIG. 13, in some configurations, computing device 1250 can include a processor 1302, a display 1304, one or more inputs 1306, one or more communication systems 1308, and/or memory 1310. In some configurations, processor 1302 can be any suitable hardware processor or combination of processors, such as a central processing unit (“CPU”), a graphics processing unit (“GPU”), and so on. In some configurations, display 1304 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 1306 can include any suitable input devices and/or sensors that can be used to receive user input, such as a keyboard, a mouse, a touchscreen, a microphone, and so on.

In some configurations, communications systems 1308 can include any suitable hardware, firmware, and/or software for communicating information over communication network 1254 and/or any other suitable communication networks. For example, communications systems 1308 can include one or more transceivers, one or more communication chips and/or chip sets, and so on. In a more particular example, communications systems 1308 can include hardware, firmware, and/or software that can be used to establish a Wi-Fi connection, a Bluetooth connection, a cellular connection, an Ethernet connection, and so on.

In some configurations, memory 1310 can include any suitable storage device or devices that can be used to store instructions, values, data, or the like, that can be used, for example, by processor 1302 to present content using display 1304, to communicate with server 1252 via communications system(s) 1308, and so on. Memory 1310 can include any suitable volatile memory, non-volatile memory, storage, or any suitable combination thereof. For example, memory 1310 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 1310 can have encoded thereon, or otherwise stored therein, a computer program for controlling operation of computing device 1250. In such configurations, processor 1302 can execute at least a portion of the computer program to present content (e.g., images, user interfaces, graphics, tables), receive content from server 1252, transmit information to server 1252, and so on. For example, the processor 1302 and the memory 1310 can be configured to perform the methods described herein, including controlling the MRI system 1100 to acquire MRI data, such as DWI data.

In some configurations, server 1252 can include a processor 1312, a display 1314, one or more inputs 1316, one or more communications systems 1318, and/or memory 1320. In some configurations, processor 1312 can be any suitable hardware processor or combination of processors, such as a CPU, a GPU, and so on. In some configurations, display 1314 can include any suitable display devices, such as an LCD screen, LED display, OLED display, electrophoretic display, a computer monitor, a touchscreen, a television, and so on. In some configurations, inputs 1316 can include any suitable input devices and/or sensors that can be used to receive user input, such as a keyboard, a mouse, a touchscreen, a microphone, and so on.

In some configurations, communications systems 1318 can include any suitable hardware, firmware, and/or software for communicating information over communication network 1254 and/or any other suitable communication networks. For example, communications systems 1318 can include one or more transceivers, one or more communication chips and/or chip sets, and so on. In a more particular example, communications systems 1318 can include hardware, firmware, and/or software that can be used to establish a Wi-Fi connection, a Bluetooth connection, a cellular connection, an Ethernet connection, and so on.

In some configurations, memory 1320 can include any suitable storage device or devices that can be used to store instructions, values, data, or the like, that can be used, for example, by processor 1312 to present content using display 1314, to communicate with one or more computing devices 1250, and so on. Memory 1320 can include any suitable volatile memory, non-volatile memory, storage, or any suitable combination thereof. For example, memory 1320 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1320 can have encoded thereon a server program for controlling operation of server 1252. In such configurations, processor 1312 can execute at least a portion of the server program to transmit information and/or content (e.g., data, images, a user interface) to one or more computing devices 1250, receive information and/or content from one or more computing devices 1250, receive instructions from one or more devices (e.g., a personal computer, a laptop computer, a tablet computer, a smartphone), and so on.

In some configurations, the server 1252 is configured to perform the methods described in the present disclosure. For example, the processor 1312 and memory 1320 can be configured to perform the methods described herein.

In some configurations, data source 1202 can include a processor 1322, one or more data acquisition systems 1324, one or more communications systems 1326, and/or memory 1328. In some configurations, processor 1322 can be any suitable hardware processor or combination of processors, such as a CPU, a GPU, and so on. In some configurations, the one or more data acquisition systems 1324 are generally configured to acquire data, images, or both, and can include an MRI system. Additionally or alternatively, in some configurations, the one or more data acquisition systems 1324 can include any suitable hardware, firmware, and/or software for coupling to and/or controlling operations of an MRI system. In some configurations, one or more portions of the data acquisition system(s) 1324 can be removable and/or replaceable.

Note that, although not shown, data source 1202 can include any suitable inputs and/or outputs. For example, data source 1202 can include input devices and/or sensors that can be used to receive user input, such as a keyboard, a mouse, a touchscreen, a microphone, a trackpad, a trackball, and so on. As another example, data source 1202 can include any suitable display devices, such as an LCD screen, an LED display, an OLED display, an electrophoretic display, a computer monitor, a touchscreen, a television, etc., one or more speakers, and so on.

In some configurations, communications systems 1326 can include any suitable hardware, firmware, and/or software for communicating information to computing device 1250 (and, in some configurations, over communication network 1254 and/or any other suitable communication networks). For example, communications systems 1326 can include one or more transceivers, one or more communication chips and/or chip sets, and so on. In a more particular example, communications systems 1326 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 1328 can include any suitable storage device or devices that can be used to store instructions, values, data, or the like, that can be used, for example, by processor 1322 to control the one or more data acquisition systems 1324, and/or receive data from the one or more data acquisition systems 1324; to generate images from data; present content (e.g., data, images, a user interface) using a display; communicate with one or more computing devices 1250; and so on. Memory 1328 can include any suitable volatile memory, non-volatile memory, storage, or any suitable combination thereof. For example, memory 1328 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1328 can have encoded thereon, or otherwise stored therein, a program for controlling operation of medical image data source 1202. In such configurations, processor 1322 can execute at least a portion of the program to generate images, transmit information and/or content (e.g., data, images, a user interface) to one or more computing devices 1250, receive information and/or content from one or more computing devices 1250, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A portable neonatal magnetic resonance imaging (MRI) system comprising:
a static (B₀) magnet comprising a plurality of magnetic elements arranged in a bulb array;
a gradient coil;
a patient bed; and
an imaging radiofrequency coil.

2. The portable neonatal MRI system of claim 1, wherein at least one of the B₀ magnet or the patient bed is movable relative to the other of the B₀ magnet or the patient bed.

3. The portable neonatal MRI system of claim 1, wherein the gradient coil is positioned outside or inside the B₀ magnet.

4. The portable neonatal MRI system of claim 1, wherein each of the plurality of magnetic elements is oriented within a Halbach bulb array to produce a homogeneous static magnetic field.

5. The portable neonatal MRI system of claim 1, wherein each of the plurality of magnetic elements is oriented within a Halbach bulb array to produce a functionally linear permanent magnetic field.

6. The portable neonatal MRI system of claim 1, wherein the imaging radiofrequency coil comprises a single channel spiral transmit/receive radiofrequency coil with variable winding density.

7. The portable neonatal MRI system of claim 5, further comprising a computer processor configured to control the MRI system to perform a diffusion weighted imaging sequence, wherein the diffusion weighted imaging sequence comprises:
acquiring an image with negligible diffusion weighting by applying dummy refocusing pulses; and
applying diffusion encoding along three approximately orthogonal directions using the permanent linear magnetic field and two or more switchable gradient coils.

8. The portable neonatal MRI system of claim 1, further comprising a shield positioned between the B₀ magnet and the imaging radiofrequency coil and an externally mounted radiofrequency coil, and wherein:
the imaging radiofrequency coil is configured to acquire imaging data while the externally mounted radiofrequency coil simultaneously acquires environmental signal; and
the portable neonatal MRI system is further configured to retrospectively remove the environmental signal from the imaging data.

9. A portable neonatal magnetic resonance imaging (MRI) system comprising:
a static (B₀) magnet comprising a plurality of permanent magnetic elements arranged in an array;
a gradient coil positioned outside the B₀ magnet;
a patient bed; and
an imaging radiofrequency coil.

10. The portable neonatal MRI system of claim 9, wherein at least one of the B₀ magnet or the patient bed is movable relative to the other of the B₀ magnet or the patient bed.

11. The portable neonatal MRI system of claim 9, wherein the array forms a Halbach bulb.

12. The portable neonatal MRI system of claim 11, wherein each of the plurality of permanent magnetic elements is oriented within the Halbach bulb array to produce a homogeneous permanent magnetic field.

13. The portable neonatal MRI system of claim 11, wherein each of the plurality of permanent magnetic elements is oriented within the Halbach bulb array to produce a functionally linear permanent magnetic field.

14. The portable neonatal MRI system of claim 9, wherein the imaging radiofrequency coil comprises a single channel spiral transmit/receive radiofrequency coil with variable winding density.

15. The portable neonatal MRI system of claim 13, further comprising a computer processor configured to control the MRI system to perform a diffusion weighted imaging sequence, wherein the diffusion weighted imaging sequence comprises:
acquiring an image with negligible diffusion weighting by applying dummy refocusing pulses; and
applying diffusion encoding along three approximately orthogonal directions using the permanent linear magnetic field and two or more switchable gradient coils.

16. The portable neonatal MRI system of claim 9, further comprising a shield positioned between the B₀ magnet and the imaging radiofrequency coil and an externally mounted radiofrequency coil, and wherein:
the imaging radiofrequency coil is configured to acquire imaging data while the externally mounted radiofrequency coil simultaneously acquires environmental signal; and
the portable neonatal MRI system is further configured to retrospectively remove the environmental signal from the imaging data.

17. A method for neonatal magnetic resonance imaging (MRI), wherein the method comprises:
transporting a portable neonatal MRI system to a bedside of a subject, wherein the portable neonatal MRI system comprises a controller, a permanent B₀ magnet comprising a plurality of permanent magnetic elements arranged in a Halbach bulb array, a gradient coil positioned outside the permanent B₀ magnet, a patient bed, and an imaging radiofrequency coil;
placing the subject on the patient bed and positioning the subject such that the Halbach bulb array partially surrounds a head of the subject;
controlling, by the controller, the gradient coil and the imaging radiofrequency coil to acquire MRI data of the subject; and
reconstructing the MRI data to produce images of the subject.

18. The method for neonatal MRI of claim 17, wherein the Halbach bulb array is configured to produce a permanent functionally linear magnetic field; and
wherein the MRI data of the subject comprises diffusion weighed imaging (DWI) data, and wherein acquiring the DWI data comprises:
acquiring an image with negligible diffusion weighting by applying dummy refocusing pulses; and
applying diffusion encoding along three approximately orthogonal directions using the permanent linear magnetic field and two or more switchable gradient coil.

19. The method for neonatal MRI of claim 17, wherein the portable neonatal MRI system further comprises a radiofrequency coil positioned outside of the permanent B₀ magnet and further comprising measuring environmental electromagnetic interference; and
wherein reconstructing the MRI data further comprises correcting for electromagnetic interference based on the measured environmental electromagnetic interference.

20. The method for neonatal MRI of claim 17, wherein the Halbach bulb array is configured to produce a permanent homogenous magnetic field.

21. A method for manufacturing a neonatal MRI system, wherein the method comprises:
determining a target magnetic field within a target scanning volume;
performing an optimization to determine an arrangement of a plurality of magnetic elements positioned within a Halbach bulb array, wherein the optimization comprises optimizing, within the target scanning volume, a simulated magnetic field based on the target magnetic field to determine at least one of positions each of the magnetic elements, orientations of each of the magnetic elements, sizes of each of the magnetic elements, or a total number of magnetic elements, and
arranging the plurality of magnetic elements in a bulb array according to the optimization.

22. The method for manufacturing the neonatal MRI system of claim 21, further comprising positioning a gradient coil outside the Halbach bulb array of magnetic elements.

23. The method for manufacturing a neonatal MRI system of claim 21, wherein the optimization is constrained by at least one of a total size of the bulb array, a size of each of the plurality of magnetic elements, or a total weight of the plurality of magnetic elements.

24. The method for manufacturing a neonatal MRI system of claim 21, wherein the target magnetic field is a homogeneous magnetic field within the target scanning volume.

25. The method for manufacturing a neonatal MRI system of claim 21, wherein the target magnetic field is a linear magnetic field within the target scanning volume.

26. A portable neonatal magnetic resonance imaging (MRI) system comprising:
a static (B₀) magnet comprising a plurality of magnetic elements arranged in a Halbach array configured to surround at least a portion of a neonatal patient;
a gradient coil;
a patient bed; and
an imaging radiofrequency coil.

27. The MRI system of claim 26, wherein the Halbach array forms a cylinder configured to surround at least a portion of a head of the neonatal patient.

28. The MRI system of claim 26, wherein the Halbach array forms a partial sphere configured to surround at least a portion of a head of the neonatal patient.

29. The MRI system of claim 26, wherein the Halbach array forms a bulb configured to surround at least a portion of a head of the neonatal patient.
